(19) United States
(12) Patent Application Publication
(10) Pub. No.: US 2017/0189418 A1
(43) Pub. Date: Jul. 6, 2017

(54) PHARMACEUTICAL COMPOSITION FOR TREATING LUNG CANCER

(71) Applicant: MA SHEN KAI RUEI CO., LTD., Tainan City (TW)

(72) Inventor: Po-Liang Chen, Taipei City (TW)

(21) Appl. No.: 14/984,461

(22) Filed: Dec. 30, 2015

(51) Int. Cl. A61K 31/575

(52) U.S. Cl. CPC A61K 31/575

(57) ABSTRACT

The present invention provides a pharmaceutical composition for treating lung cancer in a subject in need thereof, which comprises a first agent comprising at least a dehydroeburicoic acid (DeEA); and a second agent comprising at least an Anctin K(AnK); as well as each of them can be obtained from the extractions of a fruiting body or a mycelium of Antrodia camphorata wherein the first agent and the second agent shows synergistic effect for use in the treatment of lung cancer or to prevent or to reduce the risk of a lung cancer metastasizing, compared to administration of the first agent or the second agent alone.

### BACKGROUND OF THE INVENTION

1. Fields of the invention

The present invention relates to a pharmaceutical composition for treating lung cancer in a subject in need thereof, comprising a first agent and a second agent obtained individually from extractions of Antrodia camphorate; especially relates a pharmaceutical composition for treating lung cancer, which comprises a first agent comprising at least a dehydroeburicoic acid (DeEA) and a second agent comprising at least an Anctin K(AnK).

2. Descriptions of Related Art

The medical term “tumor” refers to the formation of a mass by abnormal cell proliferation which even violates surrounding or distant tissue, affecting the tissue's normal physiological function. The tumors are generally determined to be benign or malignant by histopathological examination. When the healthy cell is taken over, it too can replicate more abnormal cells. The malignant tumors are called cancer. It is known that many types of cancer are caused by genetic aberrations, i.e., mutations. In recent decades, cancer has been one of the top ten causes of death of the people in Taiwan.

Cancer starts when cells begin to grow out of control. Cells in nearly any part of the body can become cancer, and can spread to other areas of the body. A malignant tumor is a group of cancer cells that can grow into (invade) surrounding tissues or spread (metastasize) to distant areas of the body.

The cancer cells grow rapidly and invade surrounding tissue, the walls of nearby blood or lymphatic vessels through bloodstream or lymph system to spread to other parts of the body. The cancer cells stop moving as they are lodged in capillaries at a distant location and divide and migrate into the surrounding tissue, then cancer cells form small tumors at the new location (called micrometastases.)

The lymph system comprises lymph which contains tissue fluid and waste products, as well as immune system cells, lymph nodes which are small, bean-shaped collections of immune system cells being deemed as important in fighting infections, and lymphatic vessels which appear like small veins and are connected with lymph nodes, except that they carry a clear fluid called lymph (instead of blood). Thus, the lymph system is one important way of distribution of cancers to be spread to other parts of the body.

The lungs are a pair of cone-shaped breathing organs inside the chest. The lungs bring oxygen into the body when breathing in and send carbon dioxide out of the body when breathing out. Each lung has sections called lobes. Two tubes called bronchi lead from the trachea (windpipe) to the lungs.

The two main types of lung cancer are non-small cell lung cancer and small cell lung cancer. The types are based on the way the cells look under a microscope. Non-small cell lung cancer is much more common than small cell lung cancer.

Lung cancer is the malignant transformation and expansion of lung tissue, and is responsible for 1.3 million deaths worldwide annually. It is the most common cause of cancer-related death in men, and the second most common in women.

Current research indicates that the factor with the greatest impact on risk of lung cancer is long-term exposure to inhaled carcinogens, especially tobacco smoke. The occurrence of lung cancer in others (less than one tenth) appears to be due to a combination of genetic factors. Radon gas and air pollution may also contribute to the development of lung cancer.

Treatment and prognosis depend upon the histological type of cancer, the stage (degree of spread), and the patient's performance status. Current treatments include surgery, chemotherapy, and radiotherapy. Overall, the five-year survival rate is about 14%.

There are two main types of lung cancer categorized by the size and appearance of the malignant cells seen by a histopathologist under a microscope: non-small cell (80%) and small-cell (roughly 20%) lung cancer. This classification, although based on simple histological criteria, has very important implications for clinical management and prognosis of the disease.

The non-small cell lung cancers (NSCLC) are grouped together because their prognosis and management are roughly identical. There are three main sub-types: squamous cell lung carcinoma, adenocarcinoma and large cell lung carcinoma.

Squamous cell carcinoma, accounting for 29% of lung cancers, also starts in the larger bronchi but grows slower. The size of these tumors varies on diagnosis.

Adenocarcinoma is the most common subtype of NSCLC, accounting for 32% of lung cancers. It is a form which starts near the gas-exchanging surface of the lung. Most cases of adenocarcinoma are associated with smoking. However, among people who have never smoked (“never-smokers”), adenocarcinoma is the most common form of lung cancer. A subtype of adenocarcinoma, the bronchioloalveolar carcinoma, is more common in female never-smokers, and may have different responses to treatment.

Large cell carcinoma is a fast-growing form, accounting for 9% of lung cancers, that grows near the surface of the lung.

Small cell lung cancer (SCLC, also called “oat cell carcinoma”) is a less common form of lung cancer. It tends to start in the larger breathing tubes and grows rapidly becoming quite large. The oncogene most commonly involved is L-myc. The “oat” cell contains dense neuro secretory granules which give this an endocrine/paraneoplastic syndrome association. It is initially more sensitive to chemotherapy, but ultimately carries a worse prognosis and is often metastatic at presentation. This type of lung cancer is strongly associated with smoking.

Other types of lung cancers include carcinoid, adenoid cystic carcinoma (cylindroma) and mucoepidermoid carcinoma.

The lung is a common place for metastasis from tumors in other parts of the body. The adrenal glands, liver, brain, and bone are the most common sites of metastasis from primary lung cancer itself.

Over time the lung cancer cells can invade nearby tissues such the underarm lymph nodes or the lungs in a process known as metastasis. The stage of the lung cancer, the size of the tumor and its rate of growth are all factors which determine the type of treatment that is offered. Because lung cancer is such a major problem of occurrence of relatively aggressive development and existing treatments have limited long-term success, it generally occurs relatively slowly over years and even decades in some cases.

Treatment options include surgery to remove the tumor, drug treatment which includes chemotherapy. The prognosis and survival rate varies widely; the five year relative survival rates vary from 98% to 23% depending on the type of lung cancer that occurs. and hormonal therapy, radiation therapy and immunotherapy.

Although the use of a few therapy has clearly improved the outcome of patients with lung cancer, an effective pharmaceutical composition for treating lung cancer in a subject in need thereof is still a clinical challenge.

Medicinal plants have been used for centuries to treat a variety of diseases and maintain health before the advent of modern medicine. The accumulation and developing knowledge of the medicinal properties of plants by personal experimentation, local custom, anecdote, and folk tradition leads to the formation of numerous traditional medical systems and therapies, including traditional Chinese medicine (TCM).

Antrodia camphorata and the mycelia products therefrom or thereof possesses edible high value with various excellent functions not only in medicinal such as having anti-oxidant, antihypersensitive and immunostimulatory effects but also the capability of improving physical health by its own anticancer activity, reduced treatment-related symptoms and other side effects similar to medical efficiency of the wild fruiting bodies.

Consequently, many products made from Antrodia camphorata and/or comprising especially the active ingredient extracted form thereof such as Antrodia oil, Antrodia extraction, Antrodia combination and so on, are broadly used in various medicine, health care applications and also Antrodia camphorata and wild cattle camphor thus has been listed as one of the biological treasure in recent years by the Taiwan Government.

Antrodia camphorata is a non-mesh skirt bacteria, an endemic fungus, and grows in the internal heartwood (or the dark/humid wood surface) of Cattle camphorin the mountainous region of Taiwan, altitude 450-2000 meters mountain forest. It is also perennial mushroom fungus and only grows in the inner wall of a wood trunk decayed decades or more, or the lodging of the dead wood wet surface of a Cattle camphor, particularly Cinnamomum kanehirai.

Antrodia camphorata is rich in Triterpenoids, immunostimulatory polysaccharides such as -D-glucan polysaccharides, Adenosine, Nicotinic acid, SOD (superoxide dismutase enzymes), Steroids, Vitamin, essential minerals and other pharmaceutically active principles.

In addition, Antrodia extraction and/or Antrodia oil also contains much important nutrients to the human body, for examples, oleic acid, palmitic acid, linoleic acid, palmitoleic acid, linolenic acid, stearic acid, meat, beans Qu acid, arachidic acid, behenic acid, tetracosanoic acid, n-heptadecyl acid, n-heptadecenoic acid, vitamin A, vitamin B, vitamin E and minerals; as well as it also can inhibit tumor metastasis, reduce the incidence of coronary heart disease, improve immunity and other effects.

Thereby, Antrodia camphorata shows various excellent functions such as detoxification, hypoglycemic effects, reducing blood pressure, improving anti-cancer effect, inhibition of histamine release effect, enhancing anti-inflammatory effect; enhancing immunity, increasing cell viability, eliminating free radicals, promoting liver cell regeneration, lowering down alanine aminotransferase, and in addition to even enhancing the phagocytic capacity of macrophages as well as having the capability in improving physical health.

Although the extracts of Antrodia camphorates, and anticancer agents or compositions comprising thereof, have medical effects as described above and have been attracted attention of people widespread, it still cannot be used as a normal anti-tumor agent for lung cancer or used as sole-therapy drug for the treatment of lung cancer, due to it is still not clear exactly know what is the particular active ingredient or bioactive composition presented therein.

As to medicine and pharmacy science, there is need of a drug for treatment or amelioration of lung cancer with satisfactory effect, there is urgent need to develop a pharmaceutical composition which is capable of solving the defects of anti-lung cancer drugs existed in the prior arts and providing excellent medical effects of treating lung cancer.

### SUMMARY OF THE INVENTION

In view of defects and problems as above-mentioned, the inventors of the present application conduct several researches with respect to those problems remained in conventional technologies of prior arts.

As results, while an pharmaceutical composition or combination comprising active ingredients or components such as the dehydroeburicoic acid (DeEA) and/or Anctin K(AnK) obtained from the extraction Antrodia camphorates, is used in the treatment and/or prevention of lung cancers, it is surprising to find that unexpected excellent effects are achieved as compared to the effects offered by the prior arts of traditional anti-cancer agent.

The unexpected excellent effect include for examples, at least reducing or regulating carcinogenic activity, preventing proliferation or even reversing of cancer cells, and also treating and/or preventing cancer and tumor metastasis.

In addition to those unexpected excellent effects, it also found not only having excellent characteristics chemistry, biology, mechanical science and physical science, but also having good performance of transmittance and transportation.

Further, it is also found that the pharmaceutical composition or combination is very easy for the user uptaking, in short digestion and absorption, and can be used as drugs or adjuvant for the treatment and/or prevention of cancers, especially can inhibit the prevention and treatment of cancer with efficacy while applied to specific cancer cells.

Thus, the present invention is achieved.

Namely, according to an aspect of the present invention, provided is a pharmaceutical composition for treating lung cancer in a subject in need thereof, which comprises (1) a first agent comprising at least a dehydroeburicoic acid (DeEA) obtained from Extractions of a fruiting body or a mycelium of Antrodia camphorata; (2) a second agent comprising at least an Anctin K(AnK) obtained from the Extractions of a fruiting body or a mycelium of Antrodia camphorata; wherein the first agent and the second agent shows synergistic effect for use in the treatment of lung cancer or to treat, prevent or to reduce the risk of a lung cancer metastasizing, compared to administration of the first agent or the second agent alone.

According to another aspect of the present invention, further provided is a pharmaceutical composition as described above, wherein the first agent is a dehydroeburicoic acid (DeEA) in a pharmaceutically effective amount for use in the treatment of lung cancer or to treat, prevent or to reduce the risk of a lung cancer metastasizing; and/or the second agent is an Anctin K(AnK) in a pharmaceutically effective amount for use in the treatment of lung cancer or to treat, prevent or to reduce the risk of a lung cancer metastasizing.

Further, according to one aspect of the present invention is to provide a pharmaceutical composition as described above, wherein the first agent and the second agent are in the form of a botanical drug substance (BDS); and may be administered to a human cancer patient in any manner selected from co-administration, a daily regimen, an episodic regimen, intravenous administration, or oral administration.

Furthermore, according to another aspect of the present invention is to provide a pharmaceutical composition as described in Claim 1, wherein the dehydroeburicoic acid (DeEA) and/or Anctin K(AnK) is present in an approximate amount of between 15 mg and 20 mg, in the ratio (DeEA: AnK) within the ranges of about 15 : 20 to about 20 :15, about 25:35 to about 35:25, or about 60:70 to about 70 :60.

Additionally, according to one aspect of the present invention is to provide a pharmaceutical composition as described above, which further comprises a pharmaceutically acceptable ingredient comprising a vehicle, a carrier, a diluent or an excipient; wherein the excipient comprises an ingredient selected from the group consisting of lactose, sucrose, a mannitol, sorbitol, maize starch, wheat starch, rice starch, potato starch, gelatin and tragacanth.

Besides, according to another one aspect of the present invention is to provide pharmaceutical composition as described above, wherein further comprises at least one additive selected from the group consisting of absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, bactericides, sweeteners, solubilizers, wetting agents, and a mixture thereof.

### DETAILED DESCRIPTION OF THE PREFERRED EMBODIMENT

In order to make the spirit and content of the present invention more completely and easily to be understood, various examples of the present invention, particularly various specific embodiments are described in more detailed hereinafter.

However, a skilled person having general knowledge in this technical field pertains to the present invention, shall understand that the present invention is of course not limited to these examples only, and it is possible to achieve the invention by means of taking advantage of other features of function, efficiency or processes which are the same or equal with the present invention.

First, the descriptive instructions or definitions with respect to a term or a description word or phase particularly used in this specification are separately described below.

Unless defined otherwise, all technical and scientific terms used herein have the same meaning as is commonly understood by one of skill in the art to which the claimed subject matter belongs.

It is to be understood that the foregoing general description and the following detailed description are exemplary and explanatory only and are not restrictive of any subject matter claimed. In this application, the use of the singular includes the plural unless specifically stated otherwise.

It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described.

As used herein, a term of “treatment (or treating)” refers to implementing a preventive, curative or palliative disposal for achievement of pharmaceutical and/or physiological effects to an individual subject or a patient having a certain medical condition, symptoms, disease, disorder or an initial condition, in order to partially or completely reduce the severity, delay the occurrence process, and/or inhibit one or more symptoms of the medical condition, abnormal and/or the probability of occurrence of behavior disorders.

As used herein, a term of “effective amount” refers to while a medical drug for cancer is administered (administering, or administration) directly or indirectly in a certain amount, and an effect of reducing the number of cancer cells, or particular purpose of treating or preventing a cancer is shown.

The said “certain amount” is so called effective amount.

As used herein, “individual (subject)” or “patient (patient)” can be used interchangeably with one another. The “individual (or individual subject)” or “patient” means, including but not limited to, a human that can accept a compound and/or method for treatment.

Except as otherwise specifically stated that the “individual (or individual subject)” or “patient” may comprise males and females of both sexes. Also, a preferable individual or patient for treatment by using a pharmaceutical composition and/or a method of the present invention is preferably a human.

In this context, the value of the parameter used for defining the scope of the present invention, in essence, inevitably contain standard deviation caused due to individual test methods, and thus it is mostly expressed by an approximate value of number.

However, in particular the implementation of Examples, the value is presented as precisely as possible. In this document, “approximate (or about)” is determined by the skilled person having the usual knowledge of the present invention generally pertains to.

Generally, as used herein, “about” includes an amount that would be expected to be within experimental error. Hence “about 10 μg” means “about 10 μg” and also “10 μg”. It also refers to the actual value which falls in the range of an acceptable standard deviation including the exact amount, for example, the actual value is expressed by a ±10%, it means within a range, ±5%, ±1%, or ±0.5% of a particular value.

According to the present invention, a pharmaceutical composition for treating lung cancer in a subject in need thereof is provided. The pharmaceutical composition comprises a first agent and a second agent obtained individually from extractions of Antrodia camphorate.

According to the present invention, the first agent used in the pharmaceutical composition for treating lung cancer, may comprise, but are not limited to at least a dehydroeburicoic acid (DeEA). In this context, the “dehydroeburicoic acid (DeHBA)” generally has a chemical structure usually represented by Formula (2) as shown below, with molecular formula of C₃1H₄₈O₃ and molecular weight of 468.7.

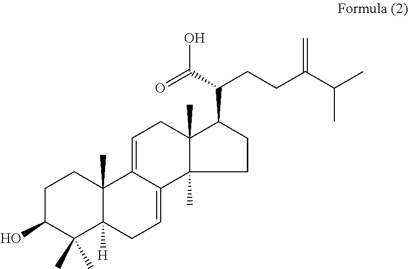

According to the present invention, dehydroeburicoic acid (DeEA) used as the first agent in the pharmaceutical composition for treating lung cancer, may be obtained by purification or isolation from the Extractions of Antrodia camphorata, which is rich in dehydroeburicoic acid (DeEA) and another bioactive ingredients for cancer treatment.

It is reported that the extractions extracted from the Antrodia camphorata comprise many effective bio-ingredients in cancer treatment, for example, Sesquiterpenoids (sesquiterpene compounds), Diterpenoids, Triterpenoids, Steroids, furan ring structure such as five member (Furan) or pyrazolyl class (Pyrrole), lignan compound (Lignoids), benzene compounds (Benzenoids), superoxide dismutase and amino acids and the like.

In the extractions extracted from the Antrodia camphorata, the Sesquiterpenoidsthat can be used as a cancer-treatment-effective bio-ingredient may comprise, but are not limited to, for example Antrocin and the like.

Further, in the extractions extracted from the Antrodia camphorata, the Diterpenoids (diterpene compounds) that can be used as a cancer-treatment-effective bio-ingredient may comprise, but are not limited to, for example 19-hydroxylabda-8(17)-en-16,15-olide, 3β,19-dihydroxylabda-8(17),11E-dien-16,15-olide, 13-epi-3β,19-dihydroxyl-abda-8(17),11E-dien-16,15-olide, 19-hydroxylabda-8(17),13-dien-6,15-olide,14-deoxy-11,12-didehydroandrograph-olide, 14-deoxy-andrographolide, pinusolidic acid and so on.

Furthermore, in the extractions extracted from the Antrodia camphorata, the Triterpenoids that can be used as a cancer-treatment-effective bio-ingredient may comprise, but are not limited to, for example CamphoratinB, camphoratin A, Antcin K, Antcin I (zhankuic acid B, 3α-hydroxy-4α-methylergost-8,24(28)-dien-7,11-dione-26-oic acid), camphoratin E, antcin H (zhankuicacid C, 3α,12α-dihydroxy-4α-methylergost-8,24(28)-dien-7,11-dione-26-oic acid), methyl antcinate H (3α,12α-dihydroxy-7,11-dioxo-4α-methylergost-8,24(28)-dien-26-oate), zhankuic acid E, camphoratin C, camphoratin H, camphoratin I, antcin A (1,4α-methylergost-8,4(8)-diene-3,11-dion-26-oic acid), camphoratin J, methyl antcinate A (methyl 4α-methylergost-8,24(28)-dien-3,11-dion-26-oate), antcin E (3,11-dioxo-4α-methylergost-8,14,24(28)-trien-26-oic acid), antcin C(7β-hydroxy-4α-methylergost-8,24(28)-diene-3,11-dion-26-oic acid), camphoratin G, antcin F (3,11-dioxo-7,8-hydroxy-4α-methylergost-8,14,24(28)-trien-26-oic acid), camphoratin D, camphoratin F, methyl antcinate G (7α-acetoxy-3,11-dioxo-4α-methylergost-8,24(28)-dien-26-oate), antcin B (zhankuicacid A, 4α-methylergost-8,24(28)-dien-3,7,11-trion-26-oic acid), antcin D (zhankuicacid F, 14-hydroxy-4α-methyl-3,7,11-trioxoergost-8,24(28)-dien-26-oic acid), methyl antcinate B (methyl 4α-methylergost-8 ,24(28)-dien-3,7,11-trion-26-oate), zhankuic acid D, eburicol (24-methylenedihydrolanosterol), eburicoic acid (35),7 sulphurenic acid, versisponic acid D, dehydroeburicoic acid, dehydrosulphurenic acid, 15α-acetyldehydrosulphurenic acid, 3β,15α-dihydroxylanosta-7,9(11),24-triene-21 oicacid, epi-friedelinol and so on.

Steroids generally comprise β-Sitosterol, stigmasterol (44),16 ergosterol peroxide, ergosterol D, ergosterol, β-sitostenone, ergosta-4,7,8(14),22-tetraen-3-one, ergo sta-2,4,8 (14),22-tetraen-3-one an so on.

Additionally, in the extractions extracted from the Antrodia camphorata, the Furan ring structures such as five (Furan) class or pyrazolyl (Pyrrole) class that can be used as a cancer-treatment-effective bio-ingredient may comprise, but are not limited to, for example Antrocinnamomin C (3-isobutyl-4-(4-hydroxyphenyl)furan-2,5-dione), 3-isobutyl-4-[4-(3-methyl-2-butenyloxy)phenyl]furan-2,5-dione antrocinnamomin D (2-hydroxy-3-isobutyl-4-[4-(3-methylbut-enyloxy) phenyl]-2H-furan-5-one), cis-3-(4-hydroxyphenyl)-4-isobutyl-dihydrofuran-2,5-dione, dimethyl-2-(4-hydroxyphenyl)-3-isobutyl-maleate, 3-(4-hydroxyphenyl)-4-isobutyl-1H-pyrrole-2,5-dione, 3-iso-utyl-4-[4-(3-methyl-2-butenyloxy) phenyl]-1Hpyrrole-2,5-dione (antrodin B, camphorataimideB), trans-3-isobutyl-4-[4-(3-methyl-2-butenyloxy)phenyl]pyrrolidine-2,5-dione, antrocinnamomin B (3-isobutyl-4-(4-hydroxyphenyl)-1H-pyrrol-1-ole-2,5-dione), 3-isobutyl-4-[4-(3-methyl-2-butenyloxy)phenyl]-1H-pyrrol-1-ol-2,5-dione (antrodin C, camphorataimide C), antrocinnamomin A(3-isobutyl-4-[4-(3-methyl-2-butenyloxy)phenyl]-1H-pyrrol-l-acetoxyl-2,5-dione), trans-1-hydroxy-3-(4-hydroxyphenyl)-4-isobutylpyrrolidine-2,5-dione, 3R,4S-1-hydroxy-3-isobutyl-4-[4-(3-methyl-2-butenyloxy) phenyl]pyrrolidine-2,5-dione), antrodin D (camphorataimide D, 3R, 4R-1-hydroxy-3-isobutyl-4-[4-(3-methyl-2-butenyloxy)phenyl]pyrrolidine-2,5-dione), antrodi-oxolanone and so on.

Besides, in the extractions extracted from the Antrodia camphorata, the Lignoids that can be used as a cancer-treatment-effective bio-ingredient may comprise, but are not limited to, for example (+)-sesamin, (−)-sesamin, 4-hydroxysesamin, Aptosimon and so on.

In the extractions extracted from the Antrodia camphorata, the Benzenoids that can be used as a cancer-treatment-effective bio-ingredient may comprise, but are not limited to, for example 1,4-dimethoxy-2,3-methylene-dioxy-5-methylbenzene, methyl 2,5-di-ethoxy-3,4-methylene-dioxybenzoate, 4,5-dimethoxy-2,3-methylene-dioxybenzoic acid, 2,4,5-trimethoxybenzaldehyde, 2,3-methylene-dioxy-6-methylbenzene-1,4-diol, 2,4-dimethoxy-6-methylbenzene-1,3-diol, benzocamphorin C, 5-methylbenzo[1,3]-dioxole-4,7-dione, 2-methoxy-5-methyl[1,4]benzo-quinone, 2,3-dimethoxy-5-methyl[1,4]benzoquinone, isobutylphenol, 2,3,4,5-tetramethoxybenzoylchloride, 2,2,5,5,-tetra-methoxy-3,4,3,4,-bis(methylenedioxy)-6,6,-dimethylbiphenyl, benzocamphorin E, benzocamphorin D, antrocamphin A, antrocamphin B, benzocamphorin A, benzocamphorin B and so on.

More specifically, in the extractions extracted from the Antrodia camphorata, the other compounds that can be used as a cancer-treatment-effective bio-ingredient may comprise, but are not limited to, for example α-Tocospiro B, methyloleate, antroquinonol, antroquinonol B, 4-acetylantroquinonol B, adenosine, cordycepin and so on.

In some embodiments, the first agent may comprise sub-ingredient which is other than the main component of dehydroeburicoic acid (DeEA) and for example, may be one compound selected form Sesquiterpenoids, Diterpenoids, Triterpenoids, Steroids, furan ring structure such as five member (Furan) or pyrazolyl class (Pyrrole), lignan compound (Lignoids), benzene compounds (Benzenoids), superoxide dismutase and amino acids and the like. Preferably, the sub-ingredient of the first agent may comprise, but are not limited to at least one selected from the group consisting of antroquinonol, antrocinnamonin A, antrocinnamonin B, antroquinonol D, zhankuic acid A, zhankuic acid C, antcin K, antcin C, and a mixture thereof.

According to the present invention, the second agent used in the pharmaceutical composition for treating lung cancer, may comprise, but are not limited to at least Anctin K(AnK). In this context, the “Anctin K(AnK)” generally has a chemical structure usually represented by Formula (1) as shown below, with molecular formula of C₂₉H₄₄O₆ and molecular weight of 488.

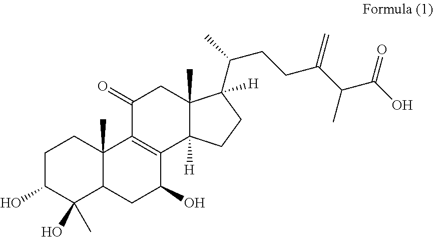 [SMILES: C=C(CC[C@@H](C)[C@H]1CC[C@H]2C3=C(C(=O)C[C@]12C)[C@@]1(C)CC[C@@H](O)[C@](C)(O)C1C[C@@H]3O)C(C)C(=O)O]

In some embodiments, the second agent may comprise Anctin K(AnK) as a main ingredient and a sub-ingredient such one compound selected form Sesquiterpenoids, Diterpenoids, Triterpenoids, Steroids, furan ring structure such as five member (Furan) or pyrazolyl class (Pyrrole), lignan compound (Lignoids), benzene compounds (Benzenoids), superoxide dismutase and amino acids and the like. Preferably, the sub-ingredient may comprise, but are not limited to at least one selected from the group consisting of antroquinonol, antrocinnamonin A, antroquinonol D, dehydrosulphurenic acid, zhankuic acid A, zhankuic acid C, antcin K, antcin C, and a mixture thereof.

According to the present invention, the extractions used for obtaining the first agent and/or the second agent of the pharmaceutical composition of the present invention, is not particularly limited, for example, can be isolated from the fruiting body or mycelium of Antrodia camphorata by using a conventional purification method well known in prior arts.

Whether raw materials are the fruiting body or mycelium of Antrodia camphorata or not, an extraction method suitable for use in general, includes non-polar solvent extraction, highly polar solvent extraction, low polar solvent extraction, high temperature extraction, lower temperature extraction method, the combination of their supercritical extraction method or the like.

For example, a solvent suitable used for extracting Antrodia camphorata typically includes water, inorganic solvents, organic solvents and the like. The organic solvents used in the present invention may include, but not limited to, alcohols such as methanol, ethanol or propanol, esters such as ethyl acetate, alkanes such as hexane, or halogenated alkanes such as chloromethane, chloroethane. Among them, water and ethanol are preferred, and ethanol is particularly preferred.

Further, extraction temperature suitably used for extracting Antrodia camphorata in the present invention is generally not particularly limited, for example, it can be conducted at below 0° C., further it also may be conducted in the lower temperature range of 0° C. to 40° C., or at a higher temperature range of above 50° C. to 150° C.

In illustrative embodiments, the Anctin K(AnK) and dehydroeburicoic acid (DeHBA) may be obtained by isolation and/or purification processes from the extractions of Antrodia camphorata. The isolation and/or purification processes used in the present invention may include, but not limited to, liquid chromatography, gas chromatography, gas-liquid chromatography, high-performance liquid chromatography (HPLC) and the likes.

Particularly, the Extraction A of Antrodia camphorata containing the first agent and/or the second agent used in the pharmaceutical composition of the present invention, was preferably obtained by by using a specific extracting method comprising steps of (A) extracting fruiting bodies of Antrodia camphorata with hot water at a temperature in a range of 45° C.-100° C. to obtain Extractions HW; (B) extracting the residues HW by a fractional distillation to obtain Extractions FD which are collected from a condensation liquid in a fractional distillation apparatus; (C) extracting the residues FD by immersing with a low polar solvent at least for 4 hours to obtain Extractions LPS; (D) extracting the residues LPS through a cryo-condensation process by dropping a iced ethanol/water with a temperature in a range of 0° C.-15° C. to obtain Extractions IEW; (E) extracting the residues IEW through a SCF (supercritical fluid extraction) by using CO₂ as a solvent at a temperature of 31.26° C. and a pressure of 72 atm to obtain Extractions SCF.

According to the present invention, the effects such as treating lung cancer, inhibiting the growth of lung cancer cell and others, of Anctin K(AnK) and/or dehydroeburicoic acid (DeHBA) may be tested by using any of the test methods used in the prior arts, for example, MTT assay of using 3-(4,5-dimethylthiazol-2-yl)-2, S-diphenyl tetrazolium bromide (MTT) to determine cell survival rates of lung cancer cell lines.

In some embodiments, through MTT assays, it is proved that Anctin K(AnK) and/or dehydroeburicoic acid (DeHBA) can decrease the survival rates of lung cancer cell lines (A549, CH27 and H460) at the same time and half-maximal inhibitory concentration (IC50) values comes to be relatively low.

Thus, the pharmaceutical composition comprising a first agent of at least a dehydroeburicoic acid (DeHBA) and a second agent of at least a Anctin K(AnK) of the present invention is very useful in inhibiting growth of preferably lung cancer cell. Also, the pharmaceutical composition of the present invention can thus be further used in preparation of a medicinal composition for treating lung cancer, which has improved the therapeutic effects with respect to the prior arts.

Further, according to one aspect of the present invention, first agent and the second agent of the pharmaceutical composition as described above, may be made to be in the form of but not limited to a botanical drug substance (BDS); and may be administered to a human cancer patient in any of manners such as the one selected from co-administration, a daily regimen, an episodic regimen, intravenous administration, or oral administration.

According to one aspect of the present invention, the effective amount of dehydroeburicoic acid (DeEA) of the first agent and Anctin K(AnK) of the second agent existed in the pharmaceutical composition as described above, may be an pharmaceutical amount useful for treating of lung cancer, or preventing or reducing the risk of a lung cancer metastasizing.

According to some embodiments of the present invention, the effective amount of dehydroeburicoic acid (DeEA) of the first agent and Anctin K(AnK) of the second agent may individually be but not limited to 0.05mg -1500.0mg. For example, it is suitable to be administrated within the range of 0.05mg -8.0 mg, preferably within the range of 0.05mg -5.50 mg, more preferably within the range of 0.05mg -4.50 mg, particularly preferably within the range of 0.05mg -2.500 mg.

Furthermore, according to one aspect of the present invention, the ratio of dehydroeburicoic acid (DeEA) and Anctin K(AnK) in the pharmaceutical composition as described above, shown as (DeEA: AnK) may be but not limited to within the ranges of about 1.0:1.0 to about 1.0:15.0. For example, it is suitable to be administrated within the range of about 1.0:1.0 to about 1.0:12.0, preferably within the range of about 1.0:1.0 to about 1.0:8.0, more preferably within the range of about 4.0:1.0 to about 1:8.0, particularly preferably within the range of about 8.0:1.0 to about 1.0:7.5.

According to some embodiments of the present invention, the effective amount of dehydroeburicoic acid (DeEA) of the first agent and Anctin K(AnK) of the second agent may be the pharmaceutical composition for lung cancer patient. Treatment the pharmaceutical composition has well effect with lung cancer patients with a favorable outcome by overexpression of the Bcl-2 gene.

Additionally, according to another one aspect of the present invention, the pharmaceutical composition may further comprises a pharmaceutically acceptable ingredient a vehicle, a carrier, a diluent or an excipient. For example, the excipient suitable used in the present invention is an ingredient, a compound or a component selected from the group consisting of lactose, sucrose, a mannitol, sorbitol, maize starch, wheat starch, rice starch, potato starch, gelatin and tragacanth, or composition or combination thereof.

Besides, according to another one aspect of the present invention, the pharmaceutical composition may further comprise an additive. For example, the additive suitable used in the present invention is at least an ingredient, a compound or a component selected from the group consisting of absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, bactericides, sweeteners, solubilizers, wetting agents, and a mixture thereof.

In some embodiments, the pharmaceutical compositions of the present invention may be formed in a liquid formulation which is suitable for use in oral administration, for example, oral suspensions, emulsions, micro-emulsions, and/or curing liquid (elixirs). In the case of the liquid formulation, the active ingredients of the pharmaceutical compositions of the present invention may be further blended with various formulations, for example, sweetening or flavoring agents, coloring matter or dyes, if necessary, it may be further blended with emulsifying and/or suspending agents, or such as water, alcohol, propylene glycol, glycerin and other diluents, or maintain buffer pH values.

Besides, in other embodiments, the formulations comprising a liquid pharmaceutical composition of the present invention may be prepared into sterile injectable solutions or suspensions; for example, it may be manufactured into a solution that is suitable for intravenous injection, intramuscular injection, it in intraperitoneal injection, or subcutaneous administration, and others.

Diluents suitable for use in a sterile injectable solution or suspension described above, for example, may include, but are not limited to, 1,3-butanediol, mannitol, water, Ringer's solution, isotonic chloride sodium; optionally natural oils or fatty acids acceptable in pharmacy, such as oleic acid, glycerol derivatives, or such as olive oil or canola oil, and the like.

The present invention is further explained in the following embodiment illustration and examples. Those examples below should not, however, be considered to limit the scope of the invention, it is contemplated that modifications will readily occur to those skilled in the art, which modifications will be within the spirit of the invention and the scope of the appended claims.

### EXAMPLES

The details of the examples for the present invention are described as follows.

### Example 1

### Extraction A Obtained from Antrodia camphorata

Firstly, an Extraction A was obtained by extracting fruiting bodies of Antrodia camphorata by using a specific method comprising steps of (A) extracting fruiting bodies of Antrodia camphorata with hot water at a temperature in a range of 45° C.-100° C. to obtain Extractions HW; (B) extracting the residues HW by a fractional distillation to obtain Extractions FD which are collected from a condensation liquid in a fractional distillation apparatus; (C) extracting the residues FD by immersing with a low polar solvent at least for 4 hours to obtain Extractions LPS; (D) extracting the residues LPS through a cryo-condensation process by dropping a iced ethanol/water with a temperature in a range of 0° C.-15° C. to obtain Extractions IEW; (E) extracting the residues IEW through a SCF (supercritical fluid extraction) by using CO₂ as a solvent at a temperature of 31.26° C. and a pressure of 72 atm to obtain Extractions SCF.

Subsequently, Extractions HW, Extractions FD, Extractions LPS, Extractions IEW and Extractions SCF were mixed uniformly to form a mixture denoted as Extraction A. In the Extraction A

### Example 2

### Ex Vivo Survival Assay for Anti-Lung Cancer Effects

The Anctin K(AnK) and dehydroeburicoic acid (DeHBA) separately isolated from the Extraction A of Example 1 were added into the culture media of human lung-cancer cells, A549, CH27 or H460, to test for tumor cell survival by using anti-cancer drug screen model. This survival assay was carried out with the widely known MTT (3-[4,5-dimethylthiazol-2-yl]2,5-diphenyltetrazolium bromide) assay.

Lung cancer cell lines, A549 cells are adenocarcinomic human alveolar basal epithelial cells. A549 cell line are widely used as an in vitro model for a type II pulmonary epithelial cell model for drug metabolism and as a transfection host, while CH27 is human lung squamous carcinoma cell and H460 is human lung non-small cell carcinoma cell. The human lung-cancer cells, A549, CH27 and H460 were cultivated in media containing fetal calf serum for 10 hours. The proliferated cells were washed once with PBS, then treated with 1× trypsin-EDTA and centrifuged at 2000 rpm for 10 minutes. The supernatant was discarded and the cell pellet was re-suspended in 200 ml of fresh culture medium by gently shaking. The cells were placed in a 96-well plate.

Subsequently, the plates were read on an ELISA reader to determine the survival rates. The half maximal inhibitory concentration (IC50) values were calculated and results of ex vivo survival assay were thus obtained.

According to those results, it shows that the lung cancer cells are capable of decreasing the survival rate of lung cancer cell A549, CH27 and H460 and namely, it is concluded that the Anctin K(AnK) and dehydroeburicoic acid (DeHBA) can inhibit the growth of lung cancer cell. On the other hand, in a pharmaceutically effective amount of the Anctin K(AnK) and dehydroeburicoic acid (DeHBA) can be applied to the treatment of lung cancer, or to prevent or to reduce the risk of a lung cancer metastasizing.

### Example 3-8

### Pharmaceutical Compositions of the Invention

The active ingredient of a first agent consisting of dehydroeburicoic acid (DeHBA) and a second agents consisting of Anctin K(AnK) separated from an Extraction A of Antrodia camphorates of Example 1, was uniformly mixed according to the composition ratio shown in table 1 to be formulated as the pharmaceutical compositions of the present invention used for a prevention and/or treatment of a lung cancer.

TABLE 1
The Pharmaceutical Compositions of The Invention

| | | First Agent | Second Agents |
|---|---|---|---|
| | | (DeHBA) | (AnK) |
| Example 3 | Composition A (wt. %) | 45 | 55 |
| Example 4 | Composition B (wt. %) | 49 | 51 |
| Example 5 | Composition C (wt. %) | 51 | 49 |
| Example 6 | Composition D (wt. %) | 30 | 70 |
| Example 7 | Composition E (wt. %) | 80 | 20 |
| Example 8 | Composition F (wt. %) | 10 | 90 |

DeHBA: dehydroeburicoic acid
AnK: Anctin K

By using the same manner as the method described above, respectively human lung cancer cell line A549, CH27 and H460 was cultured. After then, the pharmaceutical compositions A-F of the present invention was applied separately, and then MTT assay for each sample was measured to assess the inhibition effectiveness of various cancer cell activity while using pharmaceutical compositions A-F of the present invention.

According to those test results of cancer suppression activity, it confirmed that human lung cancer cell line A549, CH27 and H460 can be suppressed by the pharmaceutical compositions A-F of the present invention, particularly all of IC50 (μg/ml) are significantly better than the prior art. In summary, the results show that the pharmaceutical composition of the present invention is a potentially useful drug having pharmaceutical effectiveness in the treatment of various cancers.

What is claimed is:

1. A pharmaceutical composition for treating lung cancer in a subject in need thereof, which comprises
(1) a first agent comprising at least a dehydroeburicoic acid (DeEA) obtained from extractions of a fruiting body or a mycelium of Antrodia camphorata;
(2) a second agent comprising at least an Anctin K(AnK) obtained from the extractions of a fruiting body or a mycelium of Antrodia camphorata;
wherein the first agent and the second agent shows synergistic effect for use in the treatment of lung cancer or to prevent or to reduce the risk of a lung cancer metastasizing, compared to administration of the first agent or the second agent alone.

2. The pharmaceutical composition as described in claim 1, wherein the first agent is a dehydroeburicoic acid (DeEA) in a pharmaceutically effective amount for use in the treatment of lung cancer, or to prevent or to reduce the risk of a lung cancer metastasizing.

3. The pharmaceutical composition as described in claim 1, wherein the second agent is an Anctin K(AnK) in a pharmaceutically effective amount for use in the treatment of lung cancer, or to prevent or to reduce the risk of a lung cancer metastasizing.

4. The pharmaceutical composition as described in claim 1, wherein the the first agent and the second agent are in the form of a botanical drug substance (BDS).

5. The pharmaceutical composition as described in claim 1, wherein the dehydroeburicoic acid (DeEA) and/or Anctin K(AnK) is present in an approximate amount of between 20 mg and 15 mg.

6. The pharmaceutical composition as described in claim 1, wherein the ratio of dehydroeburicoic acid (DeEA) to Anctin K(AnK) is between about 15:20 to about 20:15 (DeEA: AnK).

7. The pharmaceutical composition as described in claim 1, wherein the ratio of dehydroeburicoic acid (DeEA) to Anctin K(AnK) is between about 25:35 to about 35:25. ((DeEA: AnK).

8. The pharmaceutical composition as described in claim 1, wherein the ratio of dehydroeburicoic acid (DeEA) to Anctin K(AnK) is between about 60:70 to about 70:60 (DeEA: AnK).

9. The pharmaceutical composition as described in claim 1, which further comprises a pharmaceutically acceptable ingredient comprising a vehicle, a carrier, a diluent or an excipient.

10. The pharmaceutical composition as described in claim 1, wherein the first agent and the second agent are administered to a human cancer patient.

11. The pharmaceutical composition as described in claim 1, wherein the first agent and the second agent are co-administered.

12. The pharmaceutical composition as described in claim 1, wherein the first agent and the second agent are administered in a daily regimen.

13. The pharmaceutical composition as described in claim 1, wherein the first agent and the second agent compositions are administered in an episodic regimen, intravenous administration, or oral administration.

14. The pharmaceutical composition as described in claim 1, wherein the excipient comprises an ingredient selected from the group consisting of lactose, sucrose, a mannitol, sorbitol, maize starch, wheat starch, rice starch, potato starch, gelatin and tragacanth.

15. The pharmaceutical composition as described in claim 1, wherein further comprises at least one additive selected from the group consisting of absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, bactericides, sweeteners, wetting agents, and a mixture thereof.

16. The pharmaceutical composition as described in claim 1, wherein the lung cancer cells are selected from the group consisting of: lung cancer cell line A549, CH27 and H460.

17. The pharmaceutical composition as described in claim 1, wherein for use in the treatment of an aggressive lung cancer characterized by overexpression of the Bcl-2 gene.
